(19) United States
(12) Patent Application Publication
(10) Pub. No.: US 2022/0370667 A1
(43) Pub. Date: Nov. 24, 2022

(54) TISSUE BOX WITH ATOMIZATION FUNCTION

(71) Applicant: Linshi Gui, Jiujiang (CN)

(72) Inventor: Linshi Gui, Jiujiang (CN)

(21) Appl. No.: 17/881,647

(22) Filed: Aug. 5, 2022

(51) Int. Cl. A61L 2/22; F21V 33/00; A47K 10/42; A61L 2/26; A61L 2/10; A45C 15/06; A45C 11/00; A45C 13/02

(52) U.S. Cl. CPC A61L 2/22; F21V 33/004; A47K 10/421; A61L 2/26; A61L 2/10; A45C 15/06; A45C 11/008; A45C 13/02; A47K 2010/3233

(57) ABSTRACT

A tissue box with an atomization function is provided, which includes a box body. A tissue chamber and a disinfection chamber are formed in the box body; the box body is also provided with a tissue outlet and a disinfection lamp; the tissue box also includes an atomization assembly and a control assembly; the atomization assembly is provided with an atomization nozzle; the box body is provided with an atomization port which is communicated with the atomization nozzle of the atomization assembly; a power supply is arranged in the box body; and the disinfection lamp, the atomization assembly and the power supply are all electrically connected to the control assembly.

### TECHNICAL FIELD

The present disclosure relates to the field of tissue boxes, specifically to a tissue box with an atomization function.

### BACKGROUND

In a tissue box, a top surface of a box body is provided with a tissue outlet and a cover for covering the tissue outlet.

A traditional tissue box only has a function of storing tissue, and this single function cannot meet the needs of modern people. Later, functions of storing toothpicks and jewelry are added on a tissue box, but this still cannot meet the needs of modern people.

Meanwhile, due to the development of the current world epidemic, people pay more attention to the hygiene of items around. People usually disinfect items by spraying or smearing alcohol on them, which is more troublesome.

### SUMMARY

The technical problem to be solved in the present disclosure is providing a tissue box with an atomization function for the above-mentioned shortcomings in the prior art, and the problems that the existing tissue box has a single function and has poor user experience are solved.

The technical solution used for solving the technical problem in the present disclosure is as follows: a tissue box with an atomization function includes:

a box body, wherein a tissue chamber used for storing tissue, and a disinfection chamber used for disinfecting items are formed in the box body; the box body is further provided with a tissue outlet communicated with the tissue chamber, and a disinfection lamp that irradiates the inside of the disinfection chamber.

The tissue box further includes an atomization assembly and a control assembly; the atomization assembly is arranged in the box body and is provided with an atomization nozzle; and the box body is provided with an atomization port which is communicated with the atomization nozzle of the atomization assembly.

The control assembly is arranged on the box body; a power supply is arranged in the box body; and the disinfection lamp, the atomization assembly and the power supply are all electrically connected to the control assembly.

According to a preferred solution, a cotton swab chamber used for storing cotton swabs is further arranged in the box body.

According to a preferred solution, two disinfection chambers are formed, one of which is arranged on an upper side of the tissue chamber and the other one of which is arranged on a lower side of the tissue chamber; the cotton swab chamber is arranged on a left side of the tissue chamber; and the atomization assembly is arranged on a right side of the tissue chamber.

According to a preferred solution, the disinfection chamber, the tissue chamber and the cotton swab chamber are all rectangular.

According to a preferred solution, the disinfection chamber is rectangular and is arranged on a short edge side of the rectangle.

According to a preferred solution, the control assembly is further electrically connected with a wireless charging module which is disposed near an upper side of the box body.

According to a preferred solution, the control assembly is further electrically connected with a lighting assembly for lighting; and the lighting assembly is provided with a lighting lamp and is disposed to irradiate an outer side of the box body.

According to a preferred solution, the control assembly is further electrically connected with a charging port used for charging an electronic product; and the charging port is exposed from one side surface of the box body.

According to a preferred solution, there are several charging ports including one or more of Micro universal serial bus (USB), Type-C, Lightning, and USB.

According to a preferred solution, the box body includes a base and a cover plate rotatably connected to the base; the tissue chamber and the disinfection chamber are both formed in the base; and the tissue outlet is arranged on the cover plate.

According to a preferred solution, one side of the cover plate is hinged to the base, and the other side of the cover plate is provided with a first fastener; and a second fastener matched with the first fastener is arranged on the base.

According to a preferred solution, the base and the cover plate are both made of an Acrylonitrile Butadiene Styrene (ABS) material.

According to a preferred solution, a fixed hole location is arranged on a back surface of the base.

According to a preferred solution, the atomization assembly includes a liquid storage bottle, a liquid pumping head and an atomization nozzle; and one end of the liquid pumping head is connected with the atomization nozzle, and the other end of the liquid pumping head is plugged into the liquid storage bottle.

According to a preferred solution, the liquid storage bottle is made of a transparent or semi-transparent material.

According to a preferred solution, the box body is detachably provided with a decorative member at the tissue outlet.

Compared with the prior art, the present disclosure has the beneficial effects that on the basis of the tissue box, the disinfection chamber and the atomization assembly are added, so that the tissue box has multiple functions; meanwhile, the cotton swab chamber is also provided to store cotton swabs; a night lamp is used during sleep at night, which further improves the integration degree of the tissue box of the present disclosure; furthermore, the tissue box of the present disclosure is particularly applicable to indoor spaces, such as a bedroom and a dining table.

Second, due to the reasonable design and ingenious arrangement of the tissue chamber, the disinfection chamber and the cotton swab chamber, the space of the box body is well utilized, and the practicability of the tissue box is improved.

### BRIEF DESCRIPTION OF THE DRAWINGS

The present disclosure is further described below in combination with the accompanying drawings and embodiments. In the drawing:

FIG. 1 is a schematic structural diagram of a tissue box without a cover plate of the present disclosure;

FIG. 2 is a schematic structural diagram of a control assembly of the present disclosure;

FIG. 3 is a schematic structural diagram of a tissue box provided with a cover plate of the present disclosure;

FIG. 4 is a schematic exploded structural diagram of a tissue box of the present disclosure;

FIG. 5 is a schematic structural diagram of a back surface of a box body of the present disclosure;

FIG. 6 is a schematic structural diagram of a disinfection lamp of the present disclosure;

FIG. 7 is a schematic structural diagram of an atomization assembly of the present disclosure;

FIG. 8 is a schematic structural diagram of a wireless charging module of the present disclosure;

FIG. 9 is a schematic structural diagram of a first winding of the present disclosure;

FIG. 10 is a schematic structural diagram of a tissue box of the present disclosure in another angle.

### DETAILED DESCRIPTION OF THE EMBODIMENTS

The technical solutions in the embodiments of the present disclosure will be clearly and completely described below in conjunction with the accompanying drawings. Apparently, the described embodiments are only preferred embodiments of the present disclosure.

It should be noted that when an element is referred to as being “fixed” to another element, it can be directly on the other element or an intermediate element may also exist. When an element is considered to be “connected” to another element, it can be directly connected to the other element or an intermediate element may be present at the same time. The terms “perpendicular”, “horizontal”, “left”, “right” and similar expressions used herein are for illustrative purposes only, and are not meant to be the only implementation modes.

Unless otherwise defined, all technical and scientific terms used herein are the same as meanings of general understandings of those skilled in the art of the present disclosure. The terms used in the description of the present disclosure herein are merely to describe the specific implementation modes, not intended to limit the present disclosure. The term “and/or” used herein includes any and all combinations of one or more related listed items.

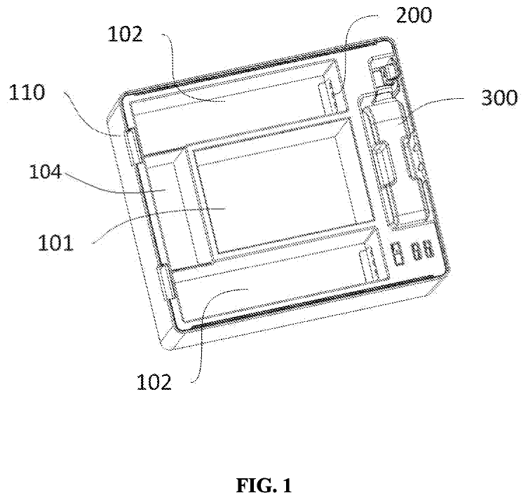

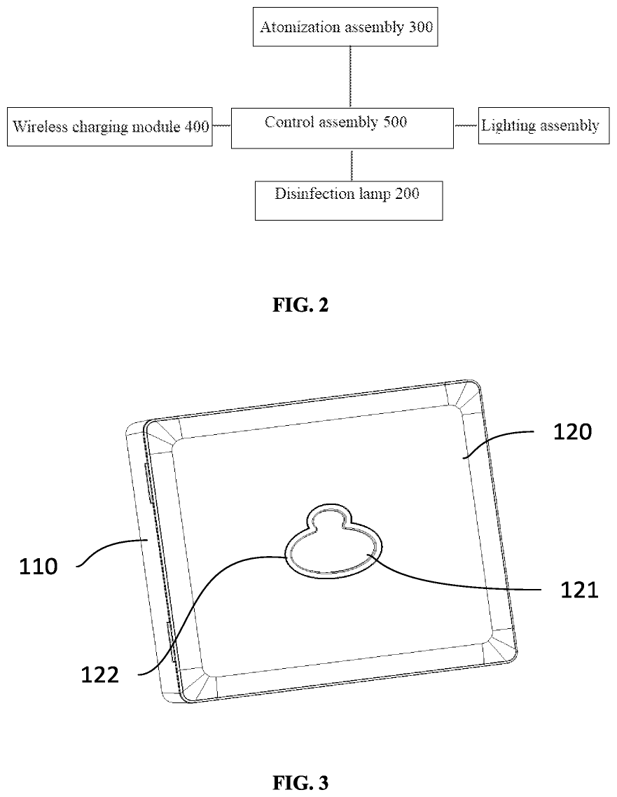

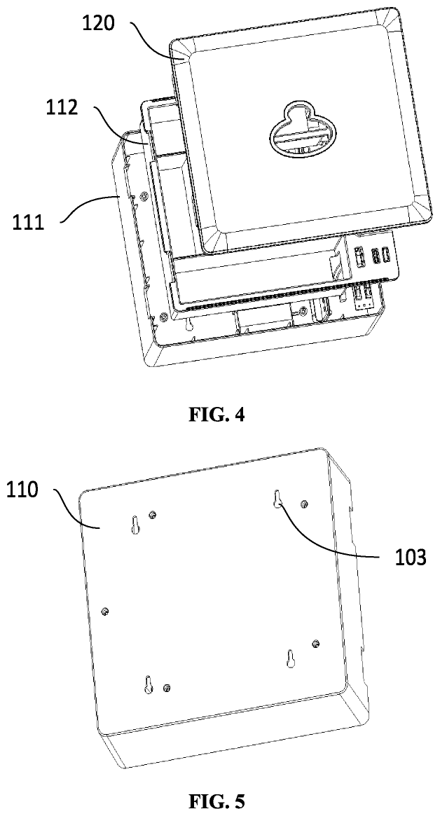

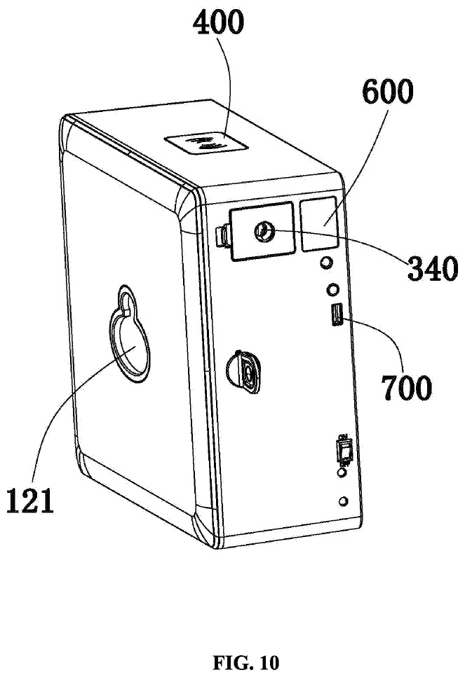

Referring to FIG. 1 to FIG. 10, a preferred embodiment of a tissue box with an atomization function in the embodiments of the present disclosure:

The tissue box includes a box body, an atomization assembly 300 and a control assembly 500. A tissue chamber 101 and a disinfection chamber 102 are at least formed in the box body; the box body also includes a tissue outlet 121 communicated with the tissue chamber 101, and a disinfection lamp 200 that irradiates the inside of the disinfection chamber 102; the atomization assembly 300 is arranged in the box body; the box body is provided with an atomization port 340. An atomization nozzle 330 of the atomization assembly 300 is communicated with the atomization port 340s; and the disinfection lamp 200 and the atomization assembly 300 are both electrically connected to the control assembly 500. Of course, the control assembly is also electrically connected with a power supply which can be a built-in storage battery of the box body. The disinfection lamp 200 is controlled to be turned on according to a control instruction or the atomization assembly 300 is controlled to work according to a control instruction.

Specifically, the tissue chamber 101 is used for storing tissue, which solves the problem of storage of tissue. Furthermore, a user fetches tissue in the tissue chamber 101 through the tissue outlet 121. At ordinary time, it can also ensure that the tissue is not contaminated. Items with suitable sizes can be placed in the disinfection chamber 102, such as a remote controller and a mobile phone, and the disinfection lamp 200 is used to irradiate the inside of the disinfection chamber 102 under a specific condition to achieve a disinfection operation. The specific condition can be automatic control or manual control. The automatic control can be carried out under a preset condition according to a preset instruction, or can be carried out after being triggered by a corresponding trigger switch or under a corresponding trigger condition. The manual control is carried out by means of a control switch, remote control, and even voice control under the assistance of assorted hardware equipment. A decorative member 122 is arranged on an inner contour of the tissue outlet 121, so that the tissue outlet 121 is made to be more attractive; and meanwhile, there can be various matchable structures, and the tissue outlet 121 can also be made to be more three-dimensional. Preferably, the decorative member 122 shall have a mounting channel with an opening facing outwards, and is snapped to an outer edge of the tissue outlet 121 through the mounting channel.

The atomization assembly 300 refers to the following: stored liquid is dispersed into tiny droplets through the atomization nozzle 330 or with an internal high-speed air flow. The multiple atomized dispersed droplets can capture granular substances in air. Methods for atomizing liquid include pressure atomization, rotating disk atomization, gas atomization, ultrasonic atomization, and the like. Liquid is transformed into droplets through a special device and is then sprayed out in the form of fog. The atomization nozzle 330 is communicated with the atomization port 340. It can be that the atomization nozzle 330 passes through the atomization port 340 and is disposed outside, or a jet tip of the atomization nozzle 330 faces the atomization port 340.

The control assembly 500 is in electrically controlled connection to the disinfection lamp 200 and the atomization assembly 300. The disinfection lamp 200 and the atomization assembly 300 are controlled according to a preset command or a corresponding control instruction, thus achieving intelligent control on the disinfection lamp 200 and the atomization assembly 300. The control assembly 500 is a main control circuit, or may be composed of a plurality of control circuits including a control logic circuit, or may achieve control using a control chip. If the control assembly is a control logic circuit, such as switch control and brightness control, corresponding trigger control is performed on the disinfection lamp 200 and the atomization assembly 300 according to a set sensor, switch or clock. If the control chip is used, the control chip and a corresponding peripheral circuit shall be included, and the control chip cooperates with the peripheral circuit according to a built-in control program to control the work of the disinfection lamp 200 and the atomization assembly 300, such as signal transmission or electric energy transmission. For example, power is supplied to the disinfection lamp 200, or a connection switch between the disinfection lamp 200 and a power supply is turned on.

The above-mentioned preset command or corresponding control instruction and the control program are simple or regular logic programs, and are not claimed in the present disclosure. What is claimed in the present disclosure is the control assembly 500 capable of controlling the work of the disinfection lamp 200 and the atomization assembly 300. The control assembly is hardware equipment, such as a control chipset, which is used as one part of a main board. The control chipset plays a key role on the main board. There are generally two control chipsets (referred to as the South Bridge and the North Bridge), which are used for controlling and harmonizing running of all components of a computer system, mainly controlling a memory I/O and a bus I/O and integrated with a hard disk controller to process an interrupt request (IRQ) and a direct memory access (DMA). For another example, a microcontroller unit (MCU), also referred to as a single chip microcomputer or a single chip computer, is a chip-level computer formed by properly reducing the frequency and specification of a central process unit (CPU) and integrating a memory, a timer, a USB, A/D conversion, a UART, a PLC, a DMA and other peripheral interfaces and even an LCD driving circuits on a single chip. Different combination control is carried out in different application occasions.

Preferably, there are two disinfection chambers 102, one of which is arranged on an upper side of the tissue chamber 101 and the other one of which is arranged on a lower side of the tissue chamber 101; the cotton swab chamber 104 used for storing cotton swabs is arranged on a left side of the tissue chamber 101, thus further adding a storage function for the tissue box of the present disclosure; and the atomization assembly 300 is arranged on a right side of the tissue chamber 101, so that due to the ingenious arrangement of the chambers on the tissue box of the present disclosure, the space utilization rate of the tissue box is increased.

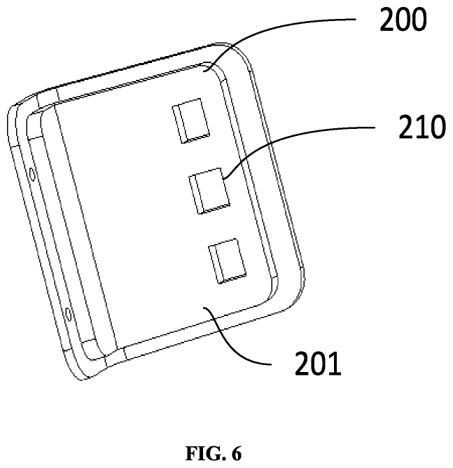

In one embodiment, referring to FIG. 6, one side wall of the disinfection chamber 102 is provided with a mounting position used for mounting the disinfection lamp 200. The disinfection lamp 200 includes a disinfection control board and at least one disinfection light emitting diode (LED) lamp bead 210 arranged on the disinfection control board. Each disinfection LED lamp bead 210 is preferably an ultraviolet LED lamp. An ultraviolet LED refers to an LED having a center wavelength of 400 nm or below. However, an LED with a light emitting wavelength greater than 380 nm is referred to as a near ultraviolet LED, and an LED with a light emitting wavelength less than 300 nm is referred to as a deep ultraviolet LED. The sterilization effect is good. The disinfection control board can be arranged in the control assembly 500, or can be disposed independently. The disinfection control board includes a circuit structure for driving the disinfection LED lamp bead 210 to be lightened and extinguished. Each disinfection LED lamp bead 210 can be arranged on a bracket 201 and then fixed in the corresponding mounting position.

Further, as shown in the figures, the tissue chamber 101, the disinfection chamber 102 and the cotton swab chamber 104 of the tissue box of this embodiment are all rectangular chambers. In this way, items are better in placement stability. More specifically, the disinfection chamber 102 is rectangular. Preferably, the disinfection lamp 200 is mounted on one side of a short edge of the rectangle. In this way, the items (such as a remote controller and a mobile phone) are disinfected more fully and thoroughly.

Preferably, the control assembly 500 is also provided with a delay closing module for controlling a work duration of the disinfection lamp 200. For example, a manufacturer can set each work duration of the disinfection lamp 200 to be 3 min. In this way, effective disinfection for the items can be guaranteed, and the problem of waste of electricity caused by the following fact can be solved: the disinfection lamp 200 is always in a work state after it has effectively disinfected the items.

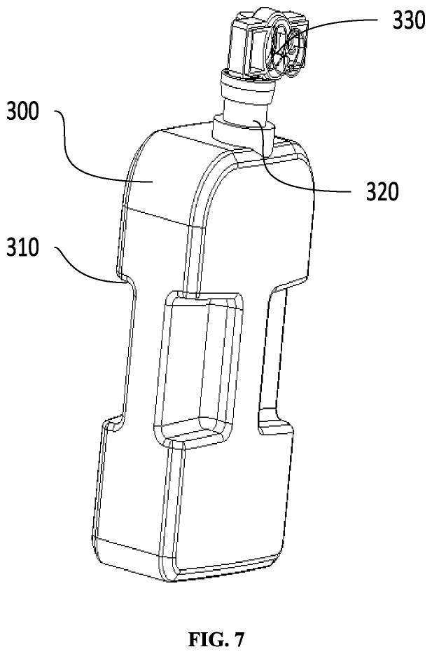

In one embodiment, referring to FIG. 7, the atomization assembly 300 includes a liquid storage bottle 310, a liquid pumping head 320 and an atomization nozzle 330. The atomization nozzle 330 atomizes liquid pumped by the liquid pumping head 320 from the liquid storage bottle 310 and sprays the atomized liquid to the outside through an atomization port. One end of the liquid pumping head 320 is connected to the atomization nozzle 330, and the other end of the liquid pumping head 320 is plugged into the liquid storage bottle 310. The liquid in the liquid storage bottle 310 enters the atomization nozzle 330 along the liquid pumping head 320 and is then sprayed to the outside through the atomization nozzle 330. Atomization can be carried out in the liquid pumping head 320, or carried out on the atomization nozzle 330. The liquid storage bottle 310 is arranged in one chamber of the box body. Clear water can be accommodated in the liquid storage bottle 310 to humidify the environment, or perfume can be accommodated in the liquid storage bottle, so as to incense the environment. It can be known from the above description that the tissue box of the present disclosure is particularly applicable to a bedside table, a dining table and other positions in a bedroom. Furthermore, the liquid storage bottle 310 is usually transparent, that is, the liquid storage bottle 310 is made of a transparent or semi-transparent material, so that it is convenient for a user to observe the content of the liquid in the liquid storage bottle 310, so as to determine whether to supplement or replace the liquid.

As shown in FIG. 3 to FIG. 5, a preferred embodiment of the box body provided in the present disclosure is as follows:

The box body includes a base 110 and a cover plate 120. The tissue chamber 101 and the disinfection chamber 102 are both arranged on the base 110, and the tissue outlet 121 is arranged on the cover plate 120; and the cover plate 120 is rotatably arranged on the base 110. Specifically, one side of the cover plate 120 is hinged to the base 110, and the other side of the cover plate 120 is provided with a first fastener. A second fastener matched with the first fastener is arranged on the base 110. In this way, the cover plate 120 can be opened and closed relative to the base 110. During use, the cover plate 120 is opened to expose the tissue chamber 101 and the disinfection chamber 102, so that tissue in the tissue chamber 101 is replaced. Or, an item to be disinfected is placed in the disinfection chamber 102, and the cover plate 120 is closed to isolate and protect the tissue in the tissue chamber 101; and at the same time, the disinfection chamber 102 is sealed for isolation to avoid ultraviolet rays of each disinfection LED lamp bead 210 from causing harm to people's health.

The base 110 of the box body includes a bottom shell 111 and a middle shell 112. The atomization port is formed in the bottom shell 111, and a back surface of the bottom shell 111 is provided with a fixed hole location 103. A plurality of chambers are formed in the middle shell 112. The tissue chamber 101 and the disinfection chamber 102 are both cavities. The liquid storage bottle 310 of the atomization assembly 300 is arranged in one cavity. The bottom shell 111 protects the middle shell 112, and the middle shell 112 forms an internal structure of the box body, such as the tissue chamber 101 and the disinfection chamber 102 as well as other accommodating structures. The back surface of the bottom shell 111 is provided with the fixed hole location 103, so that the entire tissue box is externally hung on a wall body.

Preferably, the bottom shell 111, the middle shell 112 and the cover plate 120 are all made of plastic, such as an ABS plastic material.

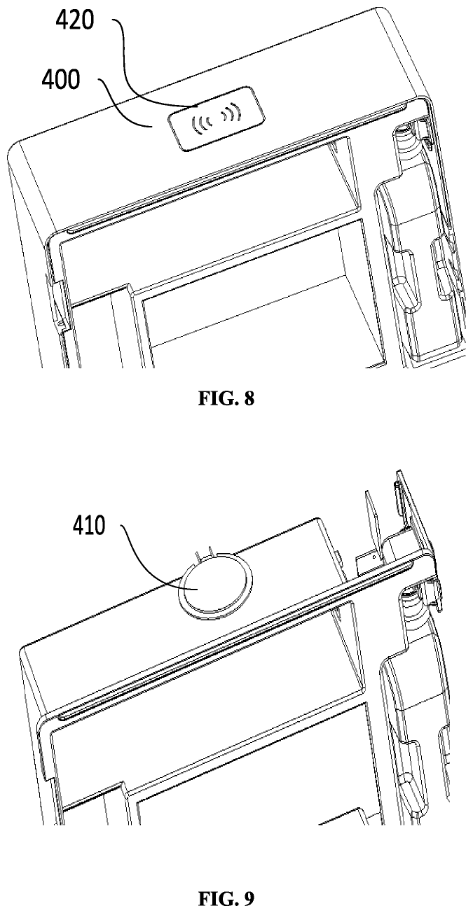

As shown in FIG. 8 to FIG. 9, the present disclosure provides a preferred embodiment of a wireless charging module 400:

The tissue box also includes a wireless charging module 400 connected to the control assembly 500. The wireless charging module 400 is controlled by the control assembly 500 to wirelessly charge a near and matching mobile terminal. Wireless charging means that a primary winding generates an alternating current with a certain frequency, and a certain current is generated in a secondary winding through electromagnetic induction, thus transferring energy from a transmission end to a receiving end. The wireless charging module 400 includes a power management circuit connected to a battery, and a first winding 410 connected to the power management circuit and serving as the primary winding. Furthermore, the first winding 410 is arranged in the box body. The power management circuit can be arranged in the control assembly 500, or can be connected to the control assembly 500 and is controlled by the control assembly 500 to control the first winding 410, and the power management circuit can also work independently. The mobile terminal is provided with a second winding serving as a secondary winding. The first winding 410 supplies power to the second winding through electromagnetic induction.

The wireless charging module 400 is arranged on an upper end surface of the box body, and the upper end surface of the box body is provided with a placement region for placing the mobile terminal. A mobile phone can be steadily placed in the placement region which shall be provided with a charging sign 420, so as to facilitate user's charging operations.

In the present disclosure, a preferred embodiment of a lighting assembly is provided:

The tissue box includes a lighting assembly. The lighting assembly irradiates the outside of the box body. Usually, the lighting assembly is used as a night lamp, which can achieve dim lighting when people take a rest at night, without affecting the sleep of people, but bringing the sense of safety to people. Preferably, the lighting assembly further includes various sensor switches, such as a human body sensor switch and a photosensitive switch, thus improving the intelligence of the lighting assembly. Furthermore, a delay closing module can be provided to control the lighting assembly, so that after the lighting assembly works for certain time, a lighting lamp 600 is automatically turned off. A specific circuit control connection method is the prior art, which will not be repeatedly described.

Further, the lighting assembly includes the lighting lamp 600 and a main ON/OFF switch which are respectively connected to the control assembly 500. The control assembly 500 controls the lighting lamp 600 to be turned on according to feedback signals of the main ON/OFF switch and the corresponding sensor switches.

In the present disclosure, a preferred embodiment of a charging port is provided:

A charging port 700 for charging electronic equipment is arranged on the tissue box. The aforementioned control assembly is electrically connected to the charging port 700. There may be several charging ports 700. Furthermore, the charging port 700 may be one or more of Micro USB, Type-C, Lightning, and USB.

Preferably, the lighting lamp 600 and the charging port 700 are arranged on the same side surface of the box body.

The design key of the present disclosure is mainly as follows: On the basis of the tissue box, the disinfection chamber and the atomization assembly are added, so that the tissue box has multiple functions; meanwhile, the cotton swab chamber is also provided to store cotton swabs; a night lamp is used during sleep at night, which further improves the integration degree of the tissue box of the present disclosure; furthermore, the tissue box of the present disclosure is particularly applicable to indoor spaces, such as a bedroom and a dining table.

Second, due to the reasonable design and ingenious arrangement of the tissue chamber, the disinfection chamber and the cotton swab chamber, the space of the box body is well utilized, and the practicability of the tissue box is improved.

The above descriptions are only preferred embodiments of the present disclosure, and do not limit the technical scope of the present disclosure. Therefore, any minor changes, equivalent changes and modifications made to the above embodiments according to the technical essence of the present disclosure still fall within the scope of the technical solutions of the present disclosure.

What is claimed is:

1. A tissue box with an atomization function, comprising:
a box body, wherein a tissue chamber used for storing tissue, and a disinfection chamber used for disinfecting items are formed in the box body; the box body is further provided with a tissue outlet communicated with the tissue chamber, and a disinfection lamp that irradiates the inside of the disinfection chamber;
the tissue box further comprises an atomization assembly and a control assembly; the atomization assembly is arranged in the box body and is provided with an atomization nozzle; the box body is provided with an atomization port which is communicated with the atomization nozzle of the atomization assembly;
the control assembly is arranged on the box body; a power supply is arranged in the box body; and the disinfection lamp, the atomization assembly and the power supply are all electrically connected to the control assembly.

2. The tissue box with the atomization function according to claim 1, wherein a cotton swab chamber used for storing cotton swabs is further arranged in the box body.

3. The tissue box with the atomization function according to claim 2, wherein two disinfection chambers are formed, one of which is arranged on an upper side of the tissue chamber and the other one of which is arranged on a lower side of the tissue chamber; the cotton swab chamber is arranged on a left side of the tissue chamber; and the atomization assembly is arranged on a right side of the tissue chamber.

4. The tissue box with the atomization function according to claim 3, wherein the disinfection chamber, the tissue chamber and the cotton swab chamber are all rectangular.

5. The tissue box with the atomization function according to claim 4, wherein the disinfection chamber is rectangular and is arranged on a short edge side of the rectangle.

6. The tissue box with the atomization function according to claim 1, wherein the control assembly is further electrically connected with a wireless charging module which is disposed near an upper side of the box body.

7. The tissue box with the atomization function according to claim 1, wherein the control assembly is further electrically connected with a lighting assembly for lighting; and the lighting assembly is provided with a lighting lamp and is disposed to irradiate an outer side of the box body.

8. The tissue box with the atomization function according to claim 1, wherein the control assembly is further electrically connected with a charging port used for charging an electronic product; and the charging port is exposed from one side surface of the box body.

9. The tissue box with the atomization function according to claim 8, wherein there are several charging ports comprising one or more of Micro universal serial bus (USB), Type-C, Lightning, and USB.

10. The tissue box with the atomization function according to claim 1, wherein the box body comprises a base and a cover plate rotatably connected to the base; the tissue chamber and the disinfection chamber are both formed in the base; and the tissue outlet is arranged on the cover plate.

11. The tissue box with the atomization function according to claim 10, wherein one side of the cover plate is hinged to the base, and the other side of the cover plate is provided with a first fastener; and a second fastener matched with the first fastener is arranged on the base.

12. The tissue box with the atomization function according to claim 10, wherein the base and the cover plate are both made of an Acrylonitrile Butadiene Styrene (ABS) material.

13. The tissue box with the atomization function according to claim 10, wherein a fixed hole location is arranged on a back surface of the base.

14. The tissue box with the atomization function according to claim 1, wherein the atomization assembly comprises a liquid storage bottle, a liquid pumping head and an atomization nozzle; and one end of the liquid pumping head is connected with the atomization nozzle, and the other end of the liquid pumping head is plugged into the liquid storage bottle.

15. The tissue box with the atomization function according to claim 14, wherein the liquid storage bottle is made of a transparent or semi-transparent material.

16. The tissue box with the atomization function according to claim 1, wherein the box body is detachably provided with a decorative member at the tissue outlet.
